(19) United States
(12) Patent Application Publication
(10) Pub. No.: US 2001/0039397 A1
(43) Pub. Date: Nov. 8, 2001

(54) Fluid delivery apparatus with flow indicator and vial fill

(76) Inventors: Marshall S. Kriesell, St.Paul, MN (US); Thomas N. Thompson, Richfield, MN (US); Steven M. Arnold, Minnetonka, MN (US); James Garrison, Minneapolis, MN (US); Rolf Hogan, Brooklyn Center, MN (US); Farhad Kazemzadeh, Bloomington, MN (US); William Kuester, Blaine, MN (US)

Correspondence Address: James E. Brunton, Suite 860, 700 North Brand Blvd., P.O. Box 29000, Glendale, CA, 91029, US

(21) Appl. No.: 09/740,096

(22) Filed: Dec. 18, 2000

Related U.S. Application Data
(63) Continuation-in-part of application No. 09/165,706, filed on Oct. 2, 1998, now Pat. No. 6,176,845.
Application No. 09/165,706 is a continuation-in-part of application No. 08/768,663, filed on Dec. 18, 1996, now Pat. No. 5,840,071.

(51) Int. Cl. A61M 37/00

(52) U.S. Cl. 604/132

(57) ABSTRACT

An apparatus for accurately infusing medicinal agents into an ambulatory patient at specific rates over extended periods of time. The apparatus includes a housing having a delivery outlet and an elastic distendable membrane for defining a chamber within the housing. Also provided is a flow rate control assembly delivery outlet. The apparatus further includes a mechanism for quickly and easily priming the fluid flow passageways of the fluid delivery component. Additionally, the apparatus includes a novel fluid flow indicator that provides a readily discernible visible indication of fluid flow through the apparatus. Further provided is a fill assembly for filling the fluid reservoir of the device with a selected medicinal fluid and locking mechanisms for preventing unauthorized tampering with the flow rate control mechanism as well as the priming mechanism.

This is a Continuation-In-Part of co-pending U.S. application Ser. No. 09/165,706 filed Oct. 2, 1998 which is a Continuation-In-Part of U.S. application Ser. No. 08/768,663 filed Dec. 18, 1996.

### BACKGROUND OF THE INVENTION

1. Field of the Invention

The present invention relates generally to fluid delivery devices. More particularly, the invention concerns an improved apparatus for infusing medicinal agents into an ambulatory patient at specific rates over extended periods of time, which apparatus includes fluid flow indicator means and a novel adjustable flow rate control means for precisely adjustably controlling the rate of fluid flow from the reservoir of the device and means for priming the device to fill the fluid passageways thereof prior to commencing the delivery step.

2. Discussion of the Prior Art

Many medicinal agents require an intravenous route for administration thus bypassing the digestive system and precluding degradation by the catalytic enzymes in the digestive tract and the liver. The use of more potent medications at elevated concentrations has also increased the need for accuracy in controlling the delivery of such drugs. The delivery device, while not an active pharmacologic agent, may enhance the activity of the drug by mediating its therapeutic effectiveness. Certain classes of new pharmacologic agents possess a very narrow range of therapeutic effectiveness, for instance, too small a dose results in no effect, while too great a dose results in toxic reaction.

In the past, prolonged infusion of fluids has generally been accomplished using gravity flow methods, which typically involve the use of intravenous administration sets and the familiar bottle suspended above the patient. Such methods are cumbersome, imprecise and require bed confinement of the patient. Periodic monitoring of the apparatus by the nurse or doctor is required to detect malfunctions of the infusion apparatus. Devices from which liquid is expelled from a relatively thick-walled bladder by internal stresses within the distended bladder are well known in the prior art. Such bladder, or “balloon” type, devices are described in U.S. Pat. No. 3,469,578, issued to Bierman and in U.S. Pat. No. 4,318,400, issued to Perry. The devices of the aforementioned patents also disclose the use of fluid flow restrictors external of the bladder for regulating the rate of fluid flow from the bladder. The prior art bladder type infusion devices are not without drawbacks. Generally, because of the very nature of the bladder or “balloon” configuration, the devices are unwieldy and are difficult and expensive to manufacture and use. Further, the devices are somewhat unreliable and their fluid discharge rates are frequently imprecise.

The apparatus of the present invention overcomes many of the drawbacks of the prior art by eliminating the bladder and making use of recently developed elastomeric films and similar materials, which, in cooperation with a base define a fluid chamber that contains the fluid which is to be dispensed. The elastomeric film membrane controllably forces fluid within the chamber into fluid flow channels provided in the base.

The elastomeric film materials used in the apparatus of the present invention, as well as various alternate constructions of the apparatus, are described in detail in U.S. Pat. No. 5,205,820 issued to the present inventor. Therefore, U.S. Pat. No. 5,205,820 is hereby incorporated by reference in its entirety as though fully set forth herein. Co-pending U.S. Ser. No. 08/768,663 filed by the present inventors on Dec. 18, 1996 also describes various alternate constructions and modified physical embodiments of the invention. Because the present application discloses improvements to the apparatus described in U.S. Ser. No. 08/768,663, this co-pending application is also hereby incorporated by reference in its entirety as though fully set forth herein. Similarly, U.S. Ser. No. 09/165,706 filed Oct. 2, 1998 filed by the present inventors describes various alternate embodiments of the invention. U.S. Pat. No. 5,721,382 issued to the present inventor on Feb. 24, 1998 discloses an apparatus for indicating fluid pressure within a conduit. The present invention comprises an improvement to the devices disclosed in this latter patent and, therefore, U.S. Pat. No. 5,721,383 is also incorporated by reference as though fully set forth herein.

The apparatus of the present invention can be used with minimal professional assistance in an alternate health care environment, such as the home. By way of example, devices of the invention can be comfortably and conveniently removably affixed to the patient's body and can be used for the continuous infusion of antibiotics, hormones, steroids, blood clotting agents, analgesics, and like medicinal agents. Similarly, the devices can be used for I-V chemotherapy and can accurately deliver fluids to the patient in precisely the correct quantities and at extended microfusion rates over time.

The embodiments of the inventions described in Ser. No. 08/768,663, and U.S. Ser. No. 09/165,706 which applications are incorporated herein by reference, concern fluid delivery devices having a fluid reservoir and an indicator assembly for indicating fluid flow through the apparatus. U.S. Ser. No. 09/165,706 also discloses a novel adjustable fluid flow rate mechanism. However, the apparatus of the present invention, includes alternate types of adjustable fluid flow rate mechanisms and also includes a novel priming mechanism for priming the device prior to commencing fluid delivery to the patient. As will be better understood from the description which follows, the novel adjustable fluid flow rate control mechanism of the present invention includes a novel type of rate control element that precisely controls the rate of fluid flow to the patient and also includes novel lockout means for disabling the priming mechanism and for locking the fluid flow rate control mechanism in a selected position.

### SUMMARY OF THE INVENTION

It is an object of the present invention to provide an apparatus for expelling fluids at a precisely controlled rate which is of a compact, low profile, laminate construction. More particularly, it is an object of the invention to provide such an apparatus which can be used for the precise infusion of pharmaceutical fluids to an ambulatory patient at controlled rates over extended periods of time.

It is another object of the invention to provide an apparatus of the aforementioned character which is highly reliable and easy-to-use by lay persons in a non-hospital environment.

Another object of the invention is to provide an apparatus, which can readily be filled in the field shortly prior to use.

A further object of the invention is to provide a low profile, fluid delivery device of laminate construction which can be manufactured inexpensively in large volume by automated machinery.

Another object of the invention is to provide a device of the aforementioned character which includes novel adjustable flow rate control means disposed intermediate the fluid reservoir outlet and the outlet port of the device for precisely controlling the rate of fluid flow from the outlet port toward the patient.

Another object of the invention is to provide a device of the character described which embodies a highly novel fluid flow indicator that provides a readily discernible visual indication of fluid flow status through the device.

Another object of the invention is to provide an apparatus of the aforementioned character which includes novel priming means for priming the device prior to commencing the fluid delivery step.

Another object of the invention is to provide unique fill means for use in controllably filling the fluid reservoir of the apparatus.

Another object of the present invention is to provide an apparatus of the aforementioned character in which the flow rate control means comprises a rotatable support disk that carries a plurality of capillary type rate control elements and is constructed and arranged so that it can be conveniently rotated by the treating physician to selectively position the rate control elements within the fluid flow path that extends between the fluid reservoir and the device outlet port.

Another object of the present invention is to provide an apparatus that includes novel means for preventing the unauthorized manipulation of the priming mechanism of the device.

Another object of the invention is to provide an apparatus as described in the preceding paragraphs which also includes fluid flow rate control locking means for locking the support disk in a preset position so that the rate control can be set only by the treating physician or an authorized health care worker having an operating key.

Another object of the invention is to provide a novel infusion means for use in delivering the medicinal fluid to the patient.

By way of summary, the improved fluid delivery apparatus of the present form of the invention comprises five major cooperating subassemblies, namely a reservoir subassembly, a highly novel adjustable, key-operated fluid flow rate control subassembly, a flow indicator subassembly for visually indicating fluid flow through the device fill means for filling the fluid reservoir and infusion means for delivering the medicinal fluid to the patient. The reservoir subassembly, which readily lends itself to automated manufacture, is generally similar to that described in copending Ser. No. 09/165,706 and includes a base and a stored energy means comprising at least one distendable elastomeric membrane which cooperates with the base to form a fluid reservoir. The fluid flow indicator subassembly is also somewhat similar to that described in Ser. No. 09/165,706 and comprises a mechanical fluid flow indicator that provides a clear visual indication of normal fluid flow and absence of fluid flow either because the reservoir is empty or because the flow lines are occluded. Additionally, the apparatus of the invention includes fill means for use in filling the reservoir of the reservoir subassembly and priming means for filling the fluid passageways of the device prior to the commencement of the fluid delivery step.

### BRIEF DESCRIPTION OF THE DRAWINGS

FIG. 1 is a generally perspective view of one form of the apparatus of the present invention which includes a flow indicator means for indicating fluid flow as well as a novel adjustable flow rate control means for precisely controlling the rate of fluid flow from the reservoir of the apparatus

FIG. 2 is a generally perspective, exploded view of the apparatus of the invention shown in FIG. 1.

FIG. 3 is a top plan view of the apparatus shown in FIG. 1.

FIG. 4 is an enlarged, side-elevational view of the apparatus illustrated in FIG. 1 shown partly in cross section to illustrate internal construction.

FIG. 5 is a greatly enlarged, front view of the delivery component of the apparatus shown in FIG. 1.

FIG. 6 is a cross-sectional view taken along lines 6-6 of FIG. 5.

FIG. 7 is a cross-sectional view taken along lines 7-7 of FIG. 5.

FIG. 8 is a view taken along lines 8-8 of FIG. 7.

FIG. 8A is a view taken along lines 8A-8A of FIG. 8.

FIG. 8B is a greatly enlarged view of the area designated as “8B” in FIG. 8A.

FIG. 9 is a fragmentary view taken along lines 9-9 of FIG. 8.

FIG. 10 is a greatly enlarged view of the area designated as “10” in FIG. 7.

FIG. 11 is a view taken along lines 11-11 of FIG. 8.

FIG. 11A is a cross-sectional view taken along lines 11A-11A of FIG. 8.

FIG. 12 is a cross-sectional view taken along lines 12-12 of FIG. 11A.

FIG. 13 is an enlarged view of the area designated as “13” in FIG. 12.

FIG. 14 is an enlarged, cross-sectional view of the central portion of FIG. 6 showing the bypass passageway of the device for bypassing the fluid flow rate control means of the invention.

FIG. 15 is a view similar to FIG. 14 but showing the priming shaft moved to a second, fluid diverting position.

FIG. 16 is a generally perspective, exploded rear view of the forward portion of the fluid delivery component of the apparatus.

FIG. 17 is a generally perspective, exploded front view of the forward portion of the fluid delivery component.

FIG. 18 is a greatly enlarged, side-elevational view of the physician's key of the invention.

FIG. 19 is a view taken along lines 19-19 of FIG. 18.

FIG. 20 is a generally schematic view showing the fluid flow path through the device during the priming step.

FIG. 20A is a generally schematic view similar to FIG. 20, but showing the fluid flow path through the device during the fluid delivery step.

FIG. 21 is an enlarged, cross-sectional view taken along lines 21-21 of FIG. 9.

FIG. 22 is a cross-sectional view taken along lines 22-22 of FIG. 21.

FIG. 23 is a cross-sectional view taken along lines 23-23 of FIG. 21.

FIG. 24 is a cross-sectional view similar to FIG. 22, but showing the key housing rotated through an angle to 90 degrees.

FIG. 25 is a cross-sectional view similar to FIG. 23, but showing the key housing rotated through 90 degrees and a locking shaft moved into a non-locking position so that the diverter shaft can be moved into its second advanced position.

FIG. 26 is an enlarged, cross-sectional view of the area designated as “26” in FIG. 24.

FIG. 27 is a cross-sectional view taken along lines 27-27 of FIG. 21.

FIG. 28 is a generally perspective, diagrammatic view of the priming shaft and a portion of the flow rate control mechanism of the apparatus of the invention and also showing a portion of the lock-out means of the apparatus of the invention in a normal lock-out configuration.

FIG. 28A is an enlarged, cross-sectional view taken along lines 28A-28A of FIG. 28.

FIG. 29 is a view similar to FIG. 28, but showing movement of the lockout means to a position permitting inward movement of the priming shaft to prime the fluid passageways of the device.

FIG. 30 is a cross-sectional view similar to FIG. 27, but showing the locking shaft having been moved into a position to permit rotation of the flow rate control knob.

FIG. 31 is also a view similar to FIG. 28, but showing movement of the lock-out means to a position shown in FIG. 30 that enables rotation of the control member of the fluid flow rate control means of the invention.

FIG. 32 is a generally perspective, exploded view of ant alternate form of flow rate control means of the invention.

FIG. 33 is a view taken along lines 33-33 of FIG. 32.

FIG. 34 is a rear view of the alternate form of the rate control means partly broken away to show internal construction.

FIG. 35 is an enlarged, cross-sectional view taken along lines 35-35 of FIG. 34.

FIG. 36 is an exploded, cross-sectional view of the assemblage shown in FIG. 35.

### DESCRIPTION OF THE INVENTION

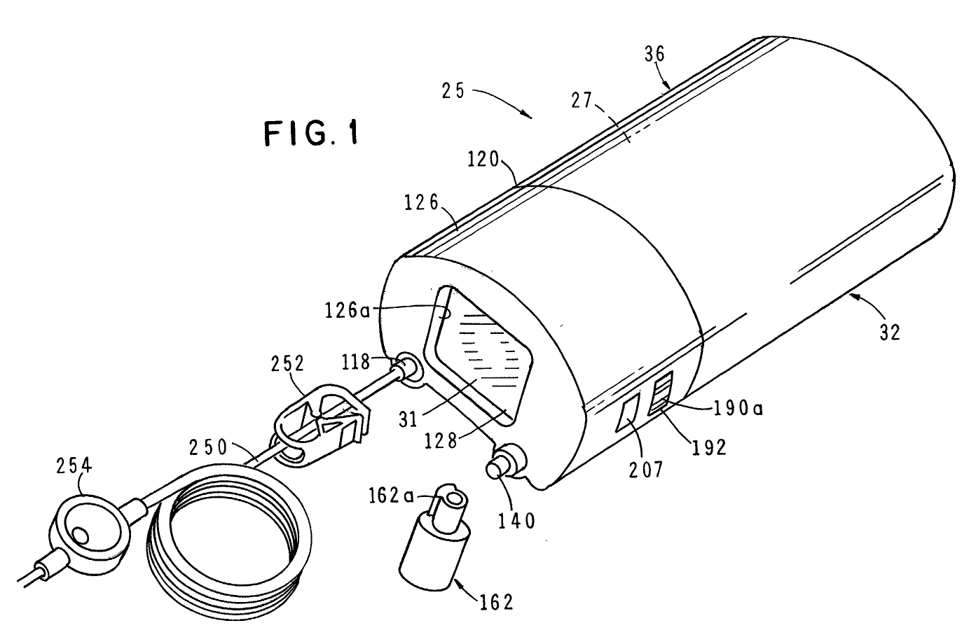

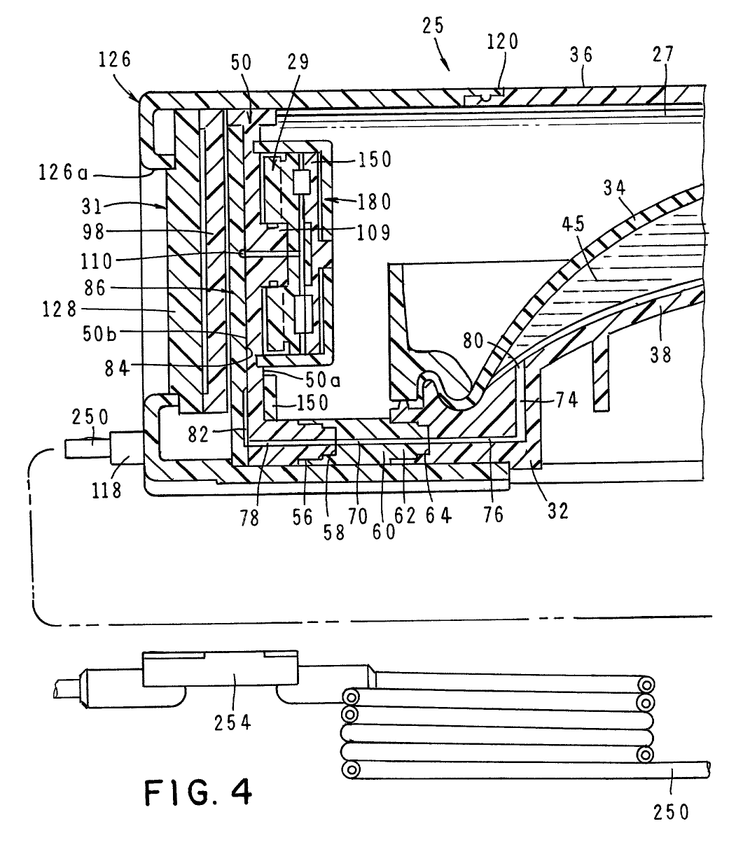

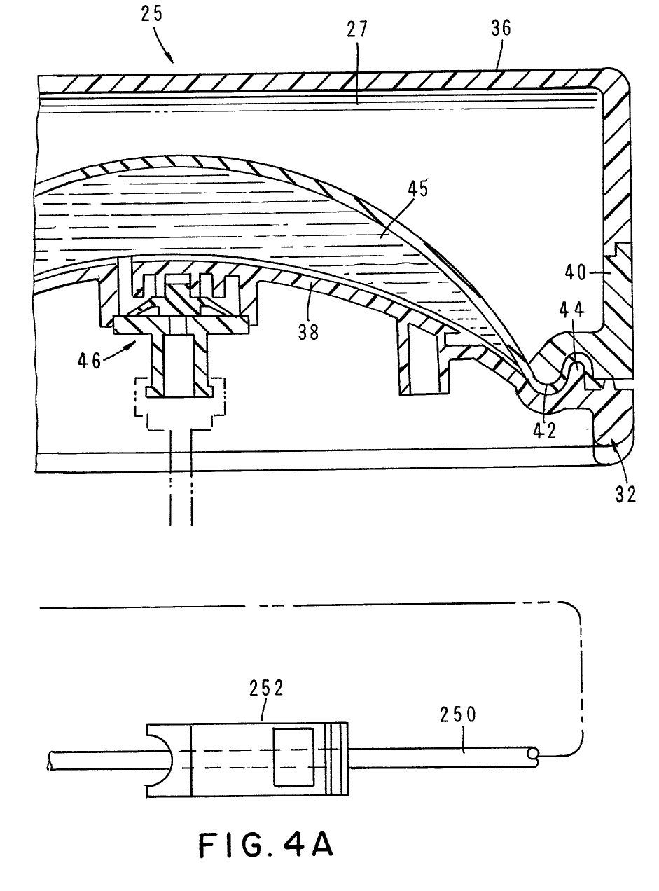

Referring to the drawings and particularly to FIGS. 1 through 4, one form of the apparatus of the invention is there illustrated and generally designated by the numeral 25. The apparatus is somewhat similar to that shown in FIGS. 26 through 37 of incorporated-by-reference Ser. No. 09/165,706 and comprises five major cooperating subassemblies namely, a reservoir subassembly 27, an adjustable flow rate control subassembly 29 (FIG. 4), a flow indicator subassembly 31, fill means for filling the fluid reservoir of the reservoir subassembly and infusion means for delivering the medicinal fluid to the patient.

Considering first the reservoir subassembly, the details of which are best seen in FIG. 4, this subassembly includes a base assembly 32, a stored energy source, shown here as a distendable membrane 34, and a cover 36 for enclosing the stored energy source. The base assembly includes an ullage substrate 38 and a membrane capture housing 40 having a bottom opening 42 which receives the distendable membrane engaging element or protuberance 44 of ullage substrate 38. Membrane 34 cooperates with ullage substrate 38 to form fluid reservoir 45. The ullage substrate, or base 38, also includes fill means, shown here as a fill assembly 46, the details of which are described in U.S. Pat. Nos. 5,962,794, 6,086,561, and 6,105,442 issued to one of the present inventors, which patents are hereby incorporated by reference as though fully set forth herein.

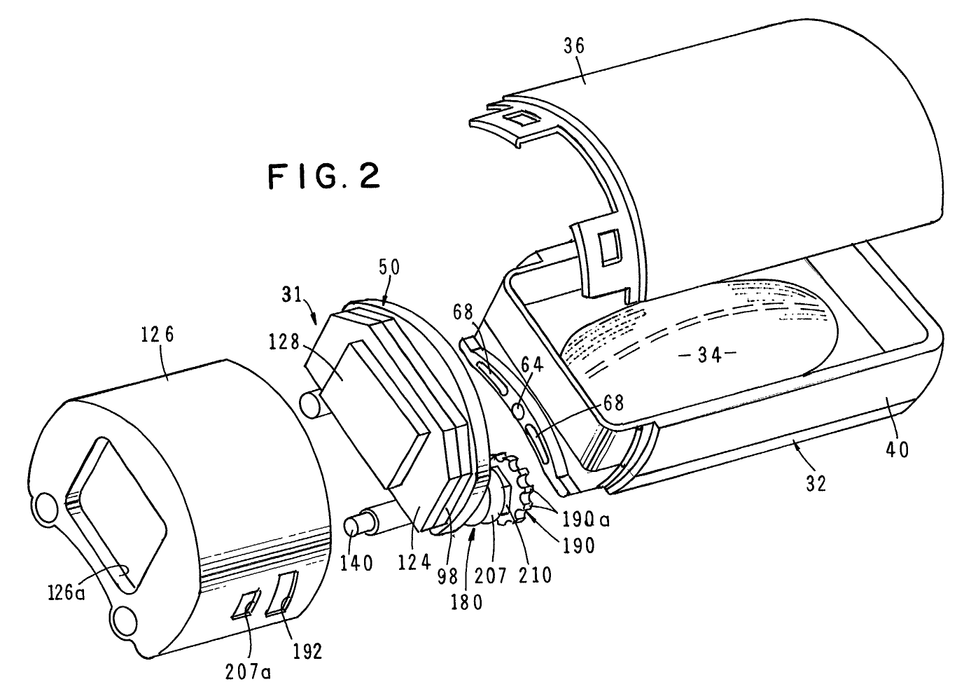

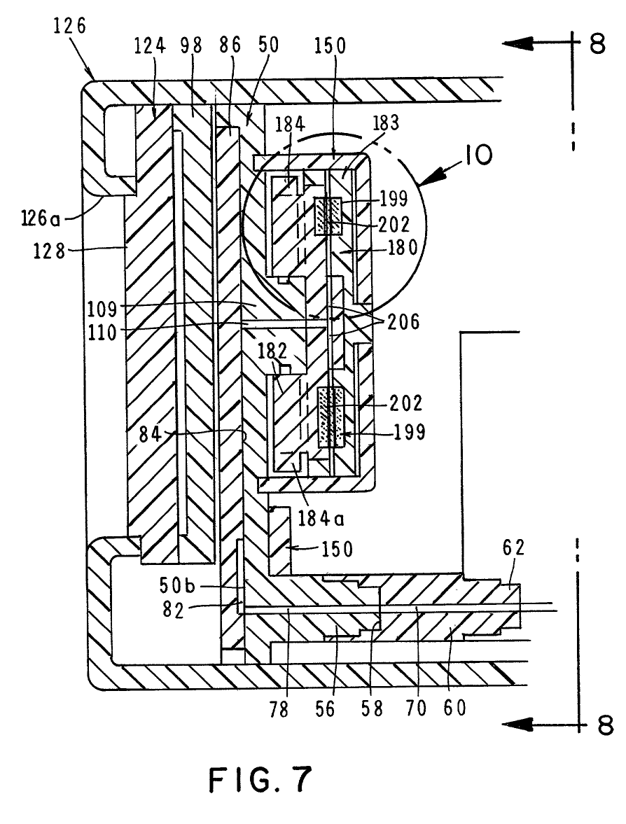

The major difference between the present embodiment of the invention and that shown in FIGS. 26 through 37 of incorporated Ser. No. 09/165,706 is the differently configured flow rate control means of the invention, which functions to precisely control the rate of fluid flow from the device. This rate control means here comprises an adjustable rate control mechanism that is carried by a support means shown here as a superstructure 50 which includes first and second faces 50a and 50b (see FIGS. 7, 16, and 17). Superstructure 50 is connected to base assembly 32 and cover 36 in the manner best seen in FIGS. 2 and 4. The details of construction of this important flow rate control means will presently be described.

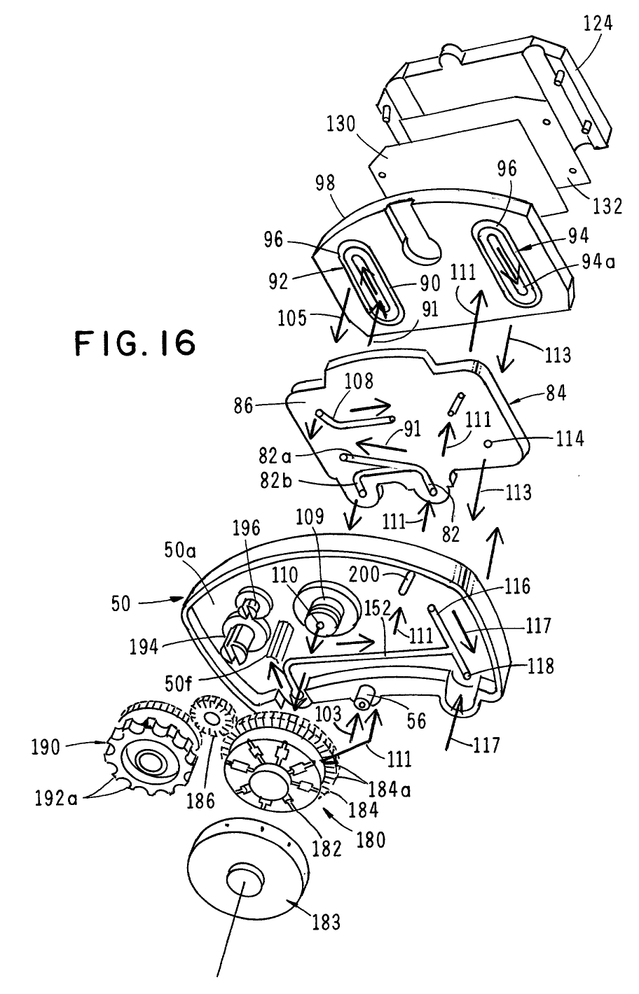

As best seen in FIG. 4, superstructure 50 of the support means includes an outwardly extending fluid inlet protuberance 56 which is closely receivable within a socket like cavity 58 formed in an extension member 60 (see also FIGS. 7, 16 and 17). Extension member 60 also has a fluid inlet protuberance 62 that is received within a socket-like cavity 64 formed in base member 32. Further, extension member 60 also has a pair of arcuate connector elements 66 (FIG. 16) that are mateably received within arcuate slots 68 formed in base assembly 32 (FIG. 2). When the support means, which includes extension member 60, is mated with base assembly 32, and connector elements 66 are received within slots 68, a fluid inlet passageway 70 formed in protuberance 62 of the extension member, is placed in fluid communication with reservoir 45 via passageways 74 and 76. Similarly, a fluid passageway 78 is formed in protuberance 56 and communicates with passageway 70 of protuberance 60 (FIG. 7).

With the construction described in the preceding paragraph, when fluid is forced through reservoir outlet 80 by the stored energy means, the fluid will flow into passageway 74, into passageway 76, into passageway 70 and then into passageway 78 formed in protuberance 56. Next, the fluid will flow into a passageway 82 formed in face 84 of a cover member 86 that is disposed in engagement with face 50b of superstructure 50 (FIGS. 4 and 16). For purposes presently to be described, passageway 82 is generally “Y” shaped having two branches 82a and 82b. As indicated by the arrows 91 in FIG. 16, during the normal fluid delivery step, branch 82a communicates with chamber 90 formed in a distendable, elastomeric first boot 92 of the flow indicator means of the invention, which is generally similar to that described in incorporated-by-reference Ser. No. 09/165,706. In addition to first boot 92, the indicator means also comprises a second boot 94 having a chamber 94a.

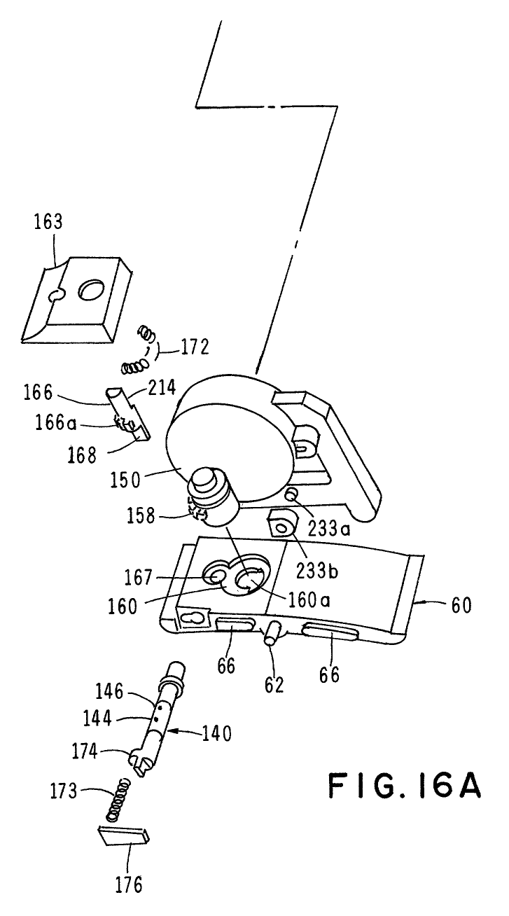

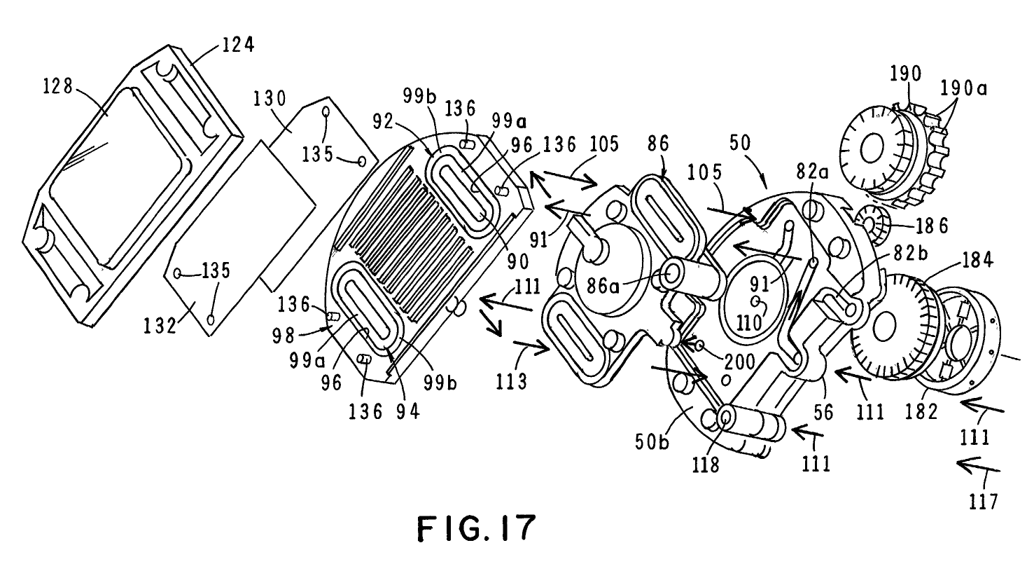

As best seen in FIG. 16, both of the boots 92 and 94 are mounted within oval shaped openings 96 formed in an indicator base 98. Boots 92 and 94 are of similar construction to boot 266 shown in FIG. 13A of incorporated-by-reference Ser. No. 08/768,663 and reference should be made to this application for a more complete discussion of the construction and operation of the flow indicator boots. As indicated in FIG. 17, each of the boots have a yieldably distendable fluid flow blocking body portion 99a which is circumscribed by a marginal portion 99b. Marginal portion 99b is clamped between cover member 86 and boot-supporting indicator base 98 so that the boot extends through the oval shaped openings 96 formed in the indicator base.

It is to be understood that the fluid flowing from reservoir 45 in the direction of arrow 103 (FIG. 16) will enter branch 82a of passageway 82 and will then flow in the direction of arrow 91 and will impinge upon boot 92. The flow will be diverted in the direction of arrows 105 of FIG. 16 and will flow rearwardly toward cover 86 and into a passageway 108 which is formed in cover 86. When cover 86 is abutted against superstructure 50, passageway 108 will communicate with an axial passageway 110 formed in hub 109 of the flow rate control mechanism. After flowing through the flow rate control means in a manner presently to be described, the fluid will flow toward boot 94 in the direction of the arrows 111 of FIG. 16. After impinging on boot 94, the fluid will be diverted in the direction of the arrows 113, through an aperture 114 formed in cover 86 and onwardly toward superstructure 50. Upon reaching superstructure 50, the fluid will flow into a passageway 116 where it will be directed in the direction of arrows 117 toward the outlet 118 of the device housing 120 with which the infusion means of the invention communicates (FIG. 1).

It is to be observed that fluid flowing from reservoir 45 toward boot 90 is under a higher pressure than fluid flowing toward boot 94. This is because the pressure of the fluid flowing toward boot 94 has been reduced as a result of the fluid flowing through rate control means of the invention. As will be discussed more fully in the paragraphs which follow, this result enables incorporation with a pair of indicator films (presently to be described) a determination of the various fluid flow operating conditions of the device namely normal fluid flow, fluid flow blockage or occlusion, and reservoir empty.

Turning particularly to FIGS. 2, 16, and 17, in addition to indicator base 98 within which boots 90 and 94 are mounted, the flow indicator means also comprises a support or lens plate 124, and a hollow forward housing 126 (FIG. 2) within which the indicator base 98 and the superstructure 50 are enclosed. As shown in FIGS. 2 and 17, a viewing lens 128 is viewable through an aperture 126a provided in forward housing 126. Disposed between indicator base 98 and lens plate 124 are first and second indicia-carrying means, which are of the character previously mentioned, and shown here as a pair of closely adjacent, thin films 130 and 132. These films are virtually identical in construction and operation to films 306 and 308 of the embodiment described in incorporated by reference Ser. No. 08/768,663 and, for a more complete understanding of the construction and operation of these films, reference should be made to this application and particularly to FIGS. 12 and 13 thereof and to the discussion of these figure drawings in the specification of the application. Films 130 and 132 are in intimate contact and are preferably constructed from a substantially transparent, flexible polymer material such as mylar. The downstream surface of the inferior or first film 130 is printed with three integrated symbols (see FIG. 12 of U.S. Ser. No. 08/768,663), which may comprise, by way of example, a blue circle, a green arrow, and a red X, each consisting of diagonal strips of color printed in an alternating pattern (blue, green, red, blue, green, red, and so on). The second film 132 serves as a “mask” over film 130 and is printed with a pattern of diagonal alternating clear and opaque strips that occur in approximately a 1:2 ratio. The printed ratio of the “mask” allows only one colored symbol to appear at a time when viewed through viewing lens 128. As in the embodiments described in U.S. Ser. No. 08/768,663, the inferior and superior films are provided at their apertures 135 which receive retention pins 136 provided on indicator base 98 (FIG. 17) which permit attachment of the films to platform 98 in a manner such that the non-patterned portions of each film covers boot openings 96a provided proximate each end of indicator base 98 with the patterned portions of both the superior and inferior films being maintained in index. With this construction, each thin film is able to move in response to pressure exerted thereon by the elastomeric boots 92 and 94 in opposing directions parallel to the film plane with its range of motion limited to one axis in the film plane by appropriate edge guides provided on indicator base 98. As more fully described in U.S. Ser. No. 08/768,663, as the films move, the visible symbol pattern will, of course, change due to the transverse displacement of the patterns imprinted thereon.

As is apparent from a study of FIGS. 13 and 13A of incorporated by reference U.S. Ser. No. 08/768,663, the central portions of both the first and second elastomeric actuator elements or boots 92 and 94 will be deflected outwardly toward plate 124 when the device is filled, but not in a state of delivery or when there is a build up of fluid pressure during delivery that is caused by blockage of the delivery line downstream from boot 94. While boot 92 can be deflected by normal line pressure, boot 94 is deflected only by pressure buildup resulting from the downstream blockage. When both elastomeric boots 90 and 94 are deflected outwardly, both the superior and inferior films are displaced transversely to a second position revealing a second symbol, as for example, an X as viewed through the viewing aperture of the support plate. When fluid is flowing through the device, an indicia such as an arrow is visable through the viewing window.

A third alignment of symbol patterns is visible when the device is in an unfilled state or when the delivery line is open, the reservoir is empty and fluid delivery to the patient has been completed. In this case, there is no fluid pressure in the line on either the upstream or the downstream side of the flow control means and thus both the first and second boots are in a non-deflected position. In this condition, the inferior and superior films are not transversely displaced and thus exhibit a third combination of patterns resulting in a third symbol as, for example, a circle being visible through the viewing aperture of the support plate. Boots 90 and 94 can be precisely tailored to deflect under various pressures thereby permitting great apparatus versatility. Reference should also be made to U.S. Ser. No. 08/432,221, which application was incorporated by reference in U.S. Ser. No. 08/768,663, for a further discussion of the construction and operation of the indicator means of the invention.

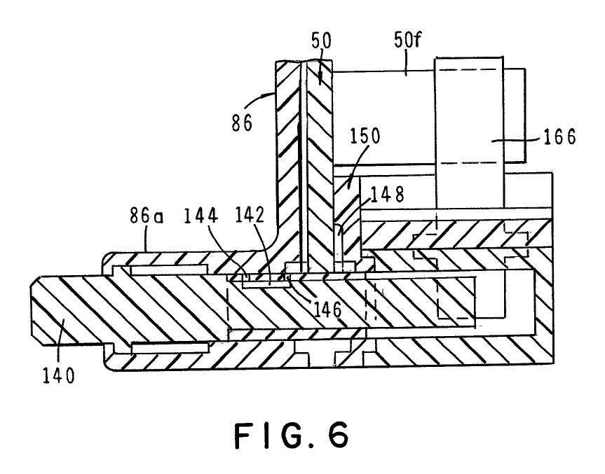

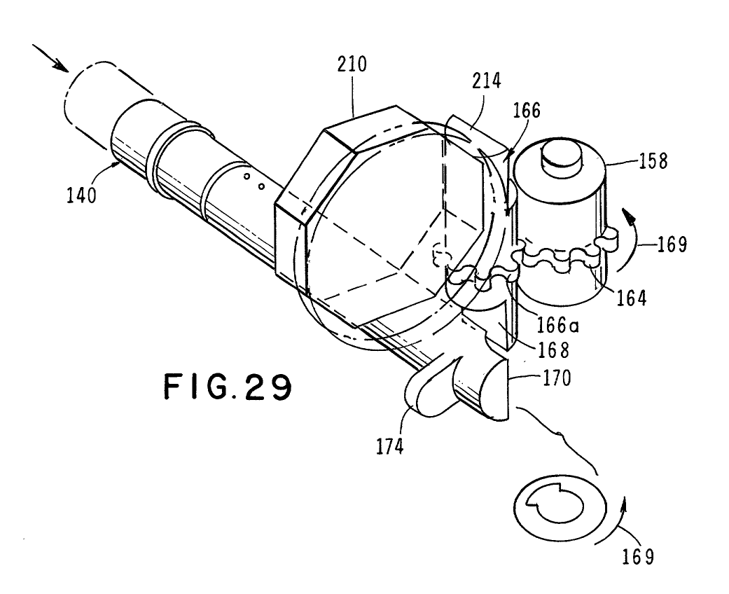

Considering next the important priming means of the invention for priming the fluid passageways of the device before commencing the fluid delivery step. This important means here comprises an elongated diverter shaft 140 that forms a part of the diverter means of the invention for causing the fluid flowing from the reservoir toward the housing or device outlet 118 to bypass the fluid flow rate control means so as to enable rapid priming of the fluid flow paths of the apparatus, including the dispenser line. In the present form of the invention, diverter shaft 140 is mounted within housing 86a in the manner best seen in FIGS. 6 and 7. As will presently be discussed, the diverter shaft can be moved by finger pressure from the at-rest, extended position shown in FIGS. 6, 14, and 28 to the advanced priming position shown in FIGS. 15 and 29.

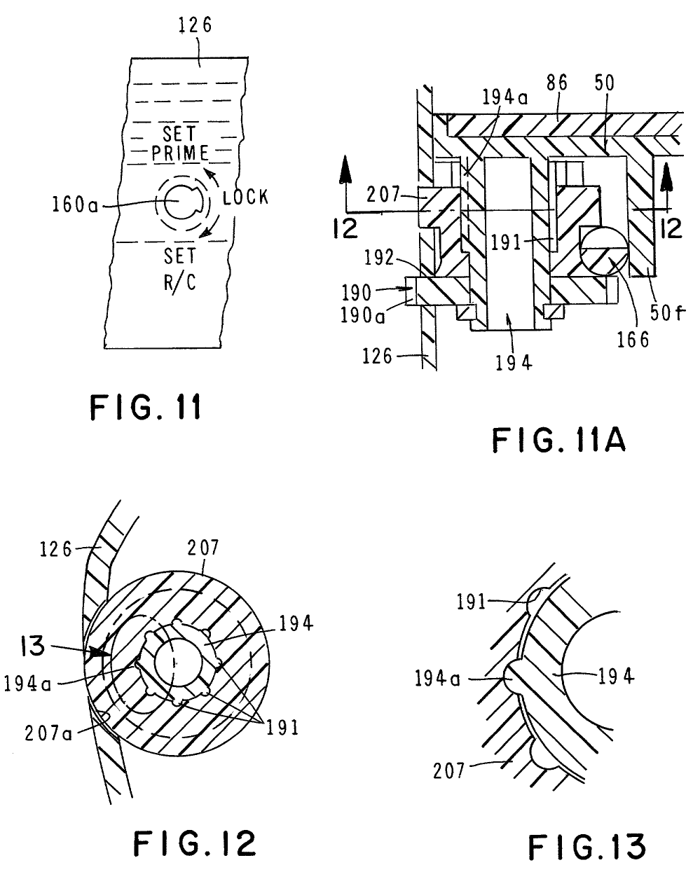

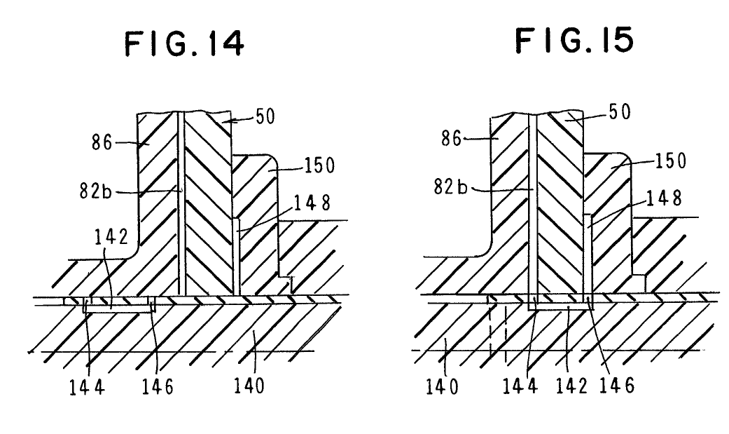

Referring particularly to FIGS. 6, 14, and 15, it is to be noted that diverter shaft 140 is provided with a fluid chamber 142 having an inlet 144 and an outlet 146 (see also FIG. 28). When shaft 140 is in the priming position shown in FIGS. 15 and 29, inlet 144 is in communication with branch 82b of passageway 82 and outlet 146 is in communication with a passageway 148 formed in a flow rate control housing and manifold 150 that is connected to superstructure 50 (FIGS. 4 and 6). With shaft 140 in its advanced or priming position, fluid can flow from reservoir 45 into branch 82b, into shaft chamber 142 and through the chamber into passageway 148. As indicated in FIG. 17, the fluid flowing into passageway 148 can then flow into the bypass passageway 152 formed in manifold 150 and into stub passageway 154 that communicates with boot 94. In this way, the various fluid passageways that comprise the fluid flow path of the device can be primed without the fluid that normally enters passageway 78 of protuberance 56 having to flow through the flow rate control means of the invention. With this novel arrangement, the time for priming the device is substantially reduced, which is essential in an ultra low flow rate device of the character described here. When the various passageways of the flow path are primed, and an inward pressure on diverter shaft 140 is removed, biasing means, shown here as spring 173, will automatically return the diverter shaft to its starting position as shown in FIG. 14 wherein fluid flow into bypass passageway 152 is blocked.

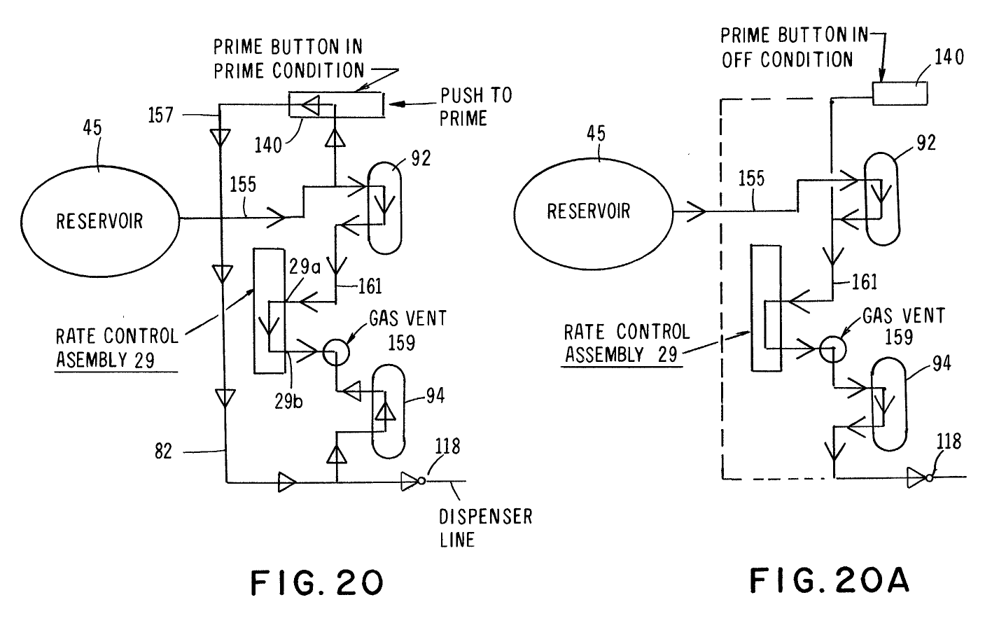

Turning to FIG. 20, which comprises a somewhat simplified schematic depiction of the fluid flow path through the device, it can be seen that during the priming step fluid will flow via a first fluid passageway segment 155 from reservoir 45 toward first boot 92 and via a second or bypass segment 157 toward second boot 94 and then onto the device outlet 118 to which the dispenser line of the infusion means is connected. As indicated in FIG. 20, fluid can also flow via a gas vent 159 toward, but not through the downstream outlet 29b of the fluid flow rate control means or rate control assembly 29. With the construction shown in FIG. 20, fluid can also flow toward the upstream inlet 29a of the rate control subassembly. Thus, as indicated in FIGS. 20, and by way of summary, fluid can flow via first segment 155 from reservoir 45 into boot 92, toward the inlet 29a of the rate control subassembly via third segment 161 and also toward diverter shaft 140. When the diverter shaft is in the prime position shown in FIG. 20, fluid can flow through the chamber 142 formed in the shaft, into bypass segment 157 toward outlet 118, toward boot 94 and also toward the outlet 29b of the rate control assembly. In this way all the fluid passageways of the device that comprise the flow path are quickly and positively primed.

Referring to FIG. 20A, it is to be noted that when the diverter shaft 140 is in its normal retracted position, as there shown, fluid can flow toward the device outlet 118 via the fluid flow rate control subassembly 29 in a normal manner. More particularly, fluid will initially flow, via first segment 155, toward first boot 92. From boot 92, fluid will flow, via third segment 161, toward the rate control assembly and then onto second boot 94 via gas vent 159. From boot 94, fluid will flow toward the device outlet 118 in the manner shown in FIG. 20A.

By way of reconciliation of FIGS. 20 and 20A with the previously described figure drawings, and in particular FIGS. 16 and 17, segment 155 as shown in FIG. 20 comprises flow passageways 74, 76, 78 (FIG. 4) and passageway 82a (FIG. 16). Similarly, second segment 157, as shown in FIG. 20, comprises passageways 82b, 152 and 116 (FIG. 16), while third segment 161 comprises passageways 108 and 110 (FIG. 16).

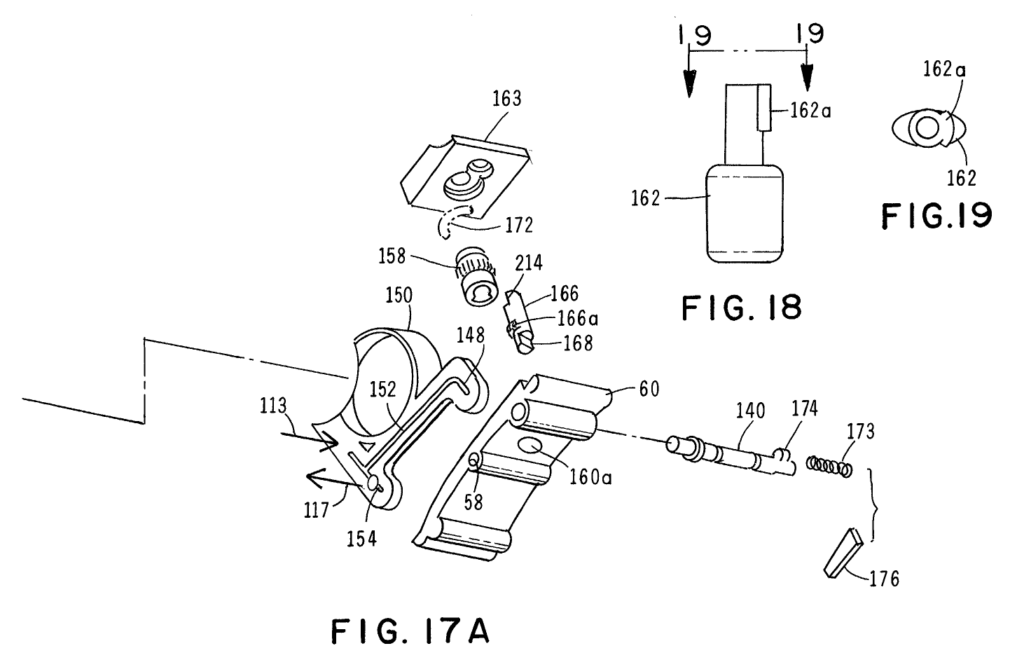

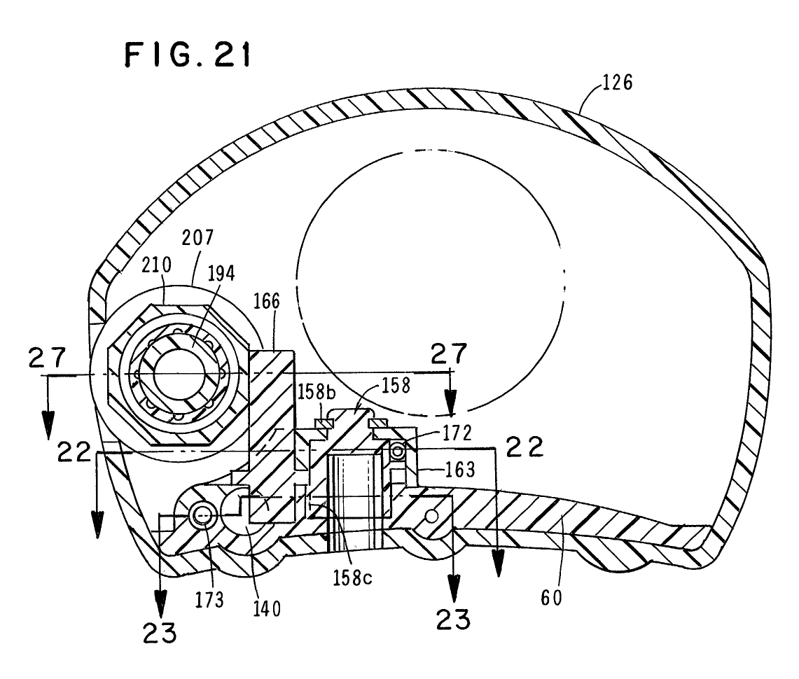

Another novel feature of the invention resides in the provision of diverter shaft locking means for preventing unauthorized advancement of diverter shaft 140 into the second priming position shown in FIGS. 15, 19, and 29. This important means here comprises a key housing 158 that is rotatably carried within a first cavity 160 formed in extension member 60 (FIG. 16) and is held in position with the device housing by a “C” shaped locking ring 158b (FIG. 21). Ring 158b engages a shoulder formed on an upper housing 163 that secures key housing 158 in place (see also FIG. 16). As shown in FIG. 23, an indexing rib 158c is receivable within a selected one of grooves 60c formed in extension 60. Cavity 160 includes a generally key-shaped opening 160a that is accessible from the bottom of extension 60 and housing 126 so that the end 162a of physician's key 162 (FIGS. 18 and 19) can be inserted into opening 160a to impart rotation to housing 158 (FIGS. 1, 11, 18, and 19).

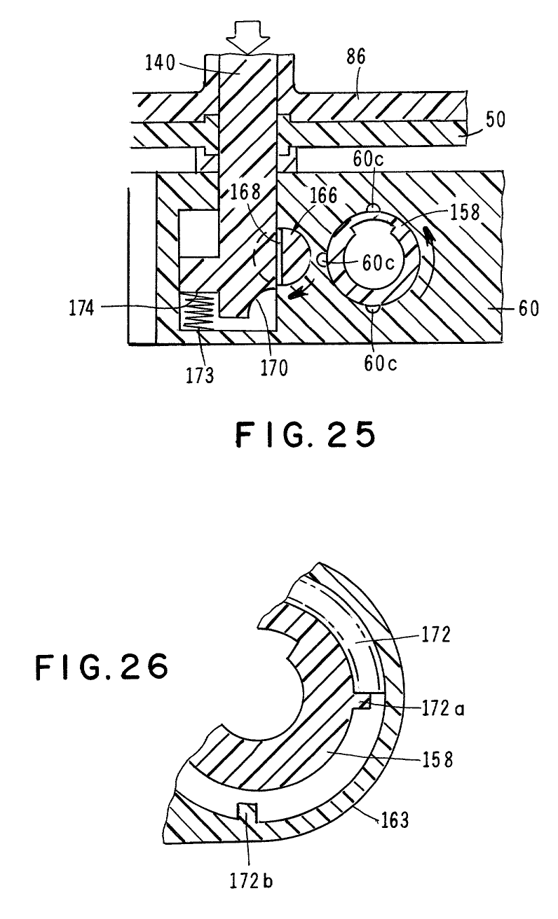

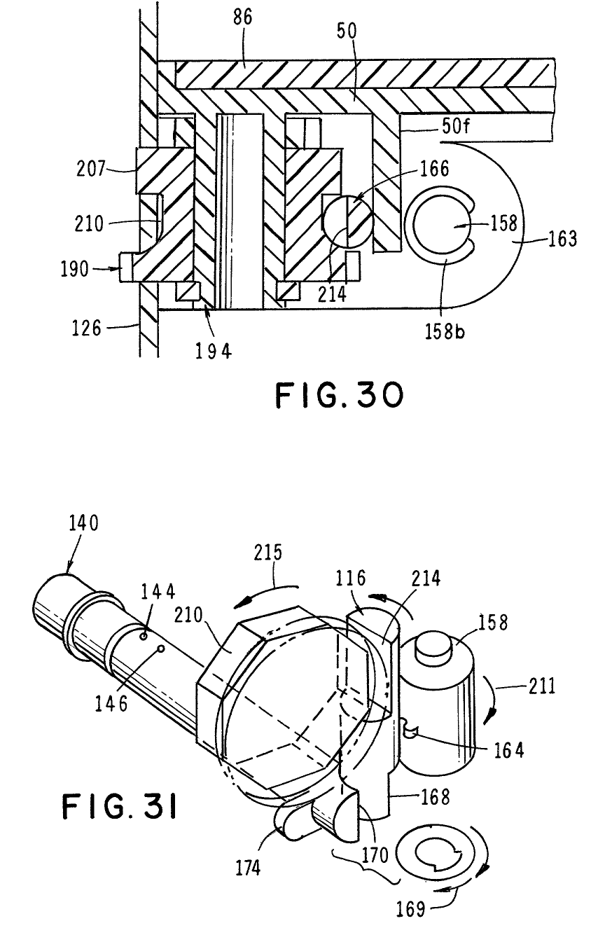

As shown in FIGS. 28 and 29, key housing 158 has a gear segment 164 that meshes with a mating gear segment 166a formed on a generally vertically extending locking shaft 166 that also comprises a part of the diverter shaft locking means. Locking shaft 166 is received within a second cavity 167 formed in extension member 60 and is positioned therewithin so that gear segment 166a meshes with the gear segment 164 of key housing 158. A finger 50f extends from superstructure 50 and serves to hold shaft 166 in position (FIGS. 16 and 30). When locking shaft 166 is in the first retracted, or normal, position shown in FIG. 28, the shaft engages a shoulder 170 formed on diverter shaft 140 (FIG. 23). However, as shown in FIGS. 24, 25, and 29, upon rotation of locking shaft 166 through an angle of 90 degrees in the direction of arrow 169 of FIG. 29, flat 168 will move into a position that will permit shoulder 170 to bypass the locking shaft so that the diverter shaft 140 can be moved into the advanced, second position shown in FIGS. 25 and 29.

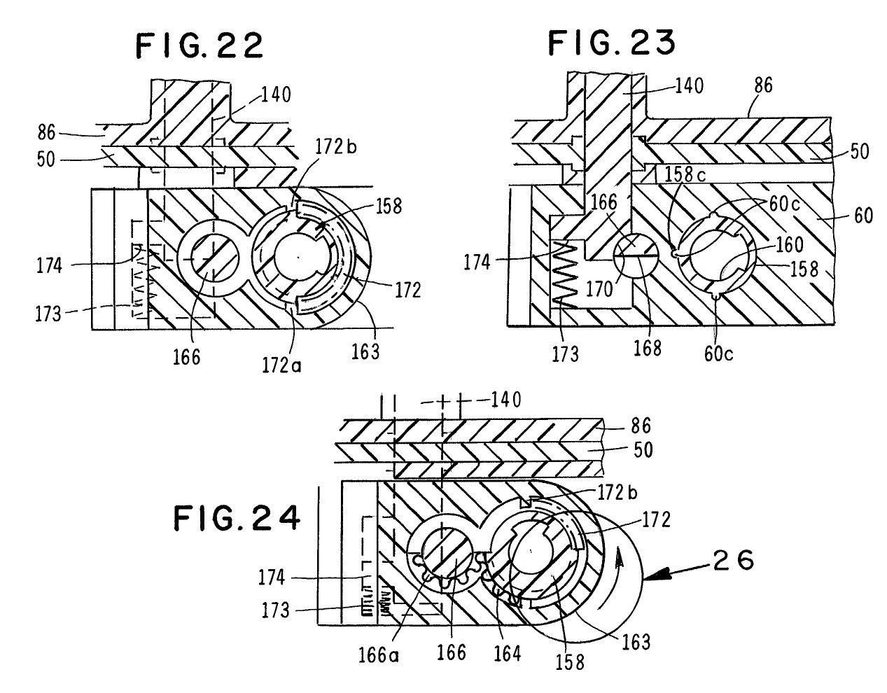

Upon release of the turning pressure exerted on the physician's key, a first biasing means shown here as an arcuate coil spring 172 which circumscribes the key housing (FIGS. 24 and 26) and is disposed between protuberances 172a and 172b (FIG. 22) will urge the key housing as well as locking shaft 166 to tend to return to their starting positions. Similarly, removal of the inward pressure exerted by the operator on the diverter shaft 140 will cause the diverter shaft to automatically return to its extended starting position due to the urging of second biasing means shown here as a coil spring 173. As illustrated in FIGS. 16, 17, 23 and 25, spring 173 is held captive between a shoulder 174 formed on the diverter shaft and an end clip 176 that is connected to extension member 60 (FIG. 16).

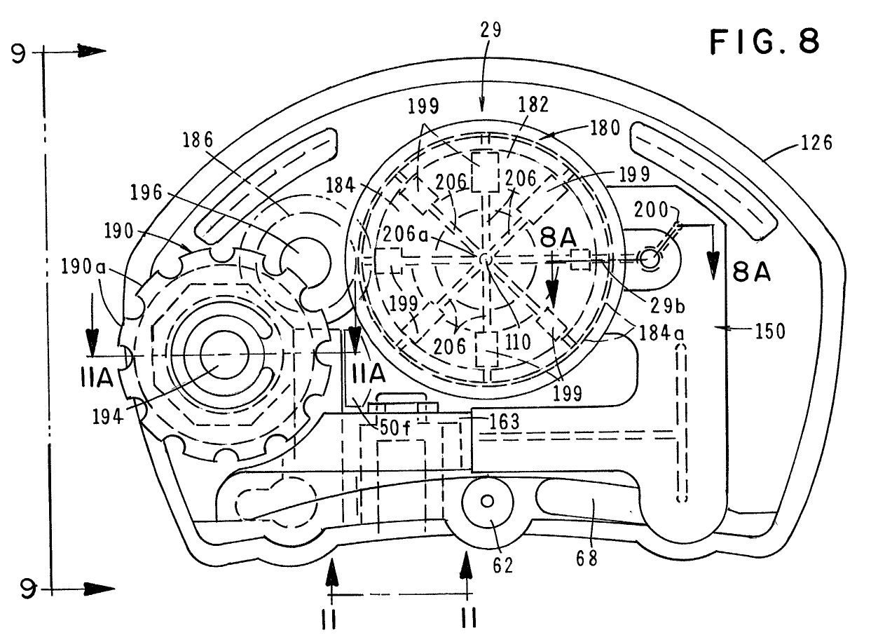

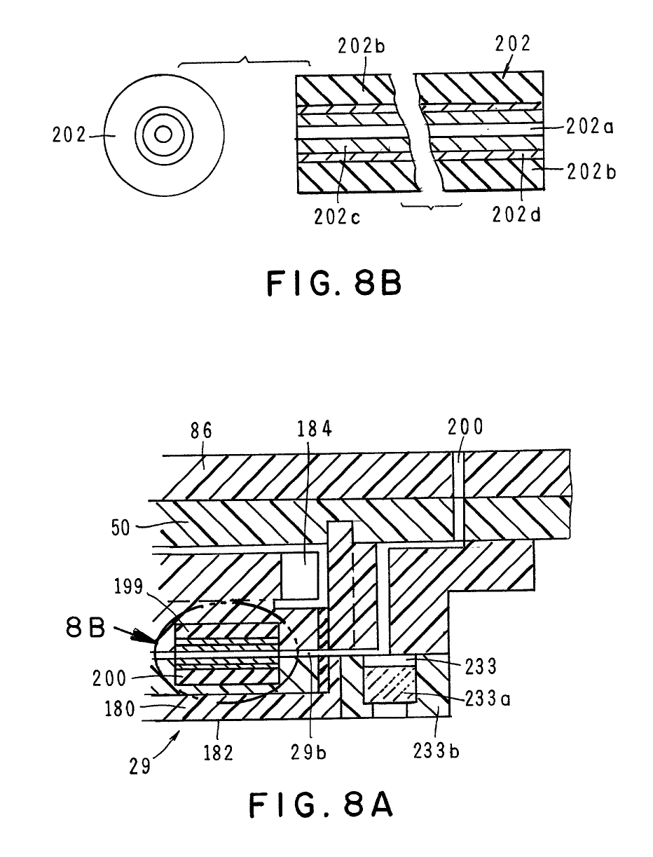

Considering next the details of the novel flow rate control means of the invention, this important means here comprises a rate control assembly 180 that is mounted for rotation on hub 109 of superstructure 50 (FIG. 16). Assembly 180 includes a rate control element base 182, a housing 183 and a back plate 184 having teeth 184a formed about its periphery (FIGS. 8, 10, and 16). Assembly 180 is controllably rotated about hub 109 by a smaller diameter driving member shown here as a toothed wheel 186 having teeth that mesh with teeth 184a. Wheel 186 is, in turn, driven by a finger engaging control knob 190 which, as shown in FIGS. 8 and 16 includes a knurled periphery 190a, a portion of which extends through an opening 192 formed in the forward housing portion 126 (FIG. 2). Control knob 109 also has teeth which mesh with toothed wheel 186 so that rotation of knob 190 about a spindle 194 (FIG. 8) formed on superstructure 50 will impart rotation to wheel 186 about a spindle 196 and will also impart rotation to assembly 180 about hub 109. Spindle 194 is provided with an indexing rib 194a that mates with a selected one of the grooves 191 provided on knob 190 so as to properly index the knob on spindle 194 (see FIGS. 11A, 12 and 13). With this construction, by rotating knob 190, a selected one of a plurality of rate control elements 199 carried by rate control element housing 182 in the manner shown in FIG. 8 can be moved into alignment with a passageway 200 of superstructure 50 (FIGS. 8 and 16) so that fluid flowing from reservoir 45 will flow therethrough at a controlled rate. After flowing through the selected rate control element, the fluid will then flow in the direction of the arrows 111 of FIG. 16 toward boot 94 and then rearwardly in the direction of the arrows 113 in the direction of device outlet 118.

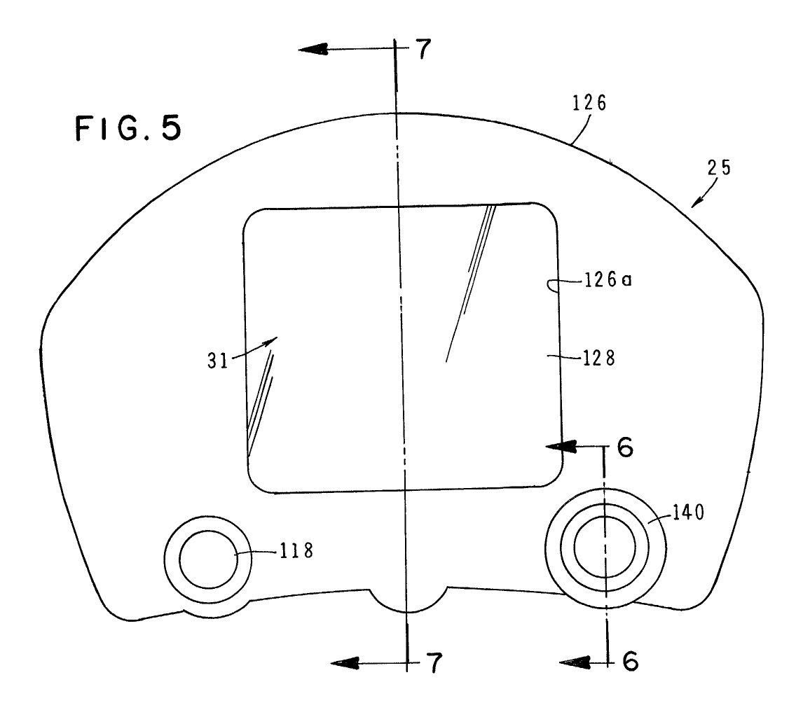

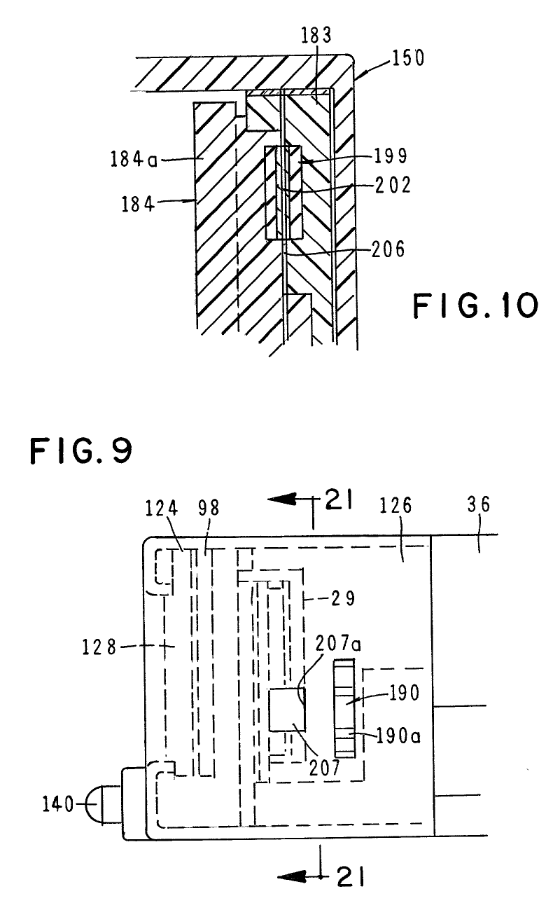

The rate control elements 199 of the flow rate control means of the invention can take several forms, but in the embodiment of the invention shown in FIGS. 1 through 31, these elements comprise glass, flow rate control capillaries 202, each having a microbore 202a of a selected size that carries the fluid through the rate control element (FIGS. 8 and 10). As best seen in FIG. 8B, capillaries 202 include an elastomer body 202b within which the glass capillary 202c is secured. Glass capillary 202c is coated with a polyimide coating 202d that is sealably connected to body 202b. Capillaries 202 are commercially available from sources such as Polymicro, Inc. of Phoenix, Ariz. Elements 199 are carried by the control member or base 182 of assembly 180 so that each communicates with a radially extending fluid flow passageway 206 formed within the assembly. Each of the passageways 206, in turn, communicates, at their inlet 206a, with passageway 110 that communicates with boot 92 in the manner previously described.

As the rate control assembly is rotated by rotation of control knob 190, a selected one of the passageways 206 containing a selected rate control element or capillary 202 is brought into fluid communication with fluid flow passageway 200. Element 202 will, of course, precisely control the rate of fluid flowing toward passageway 200 and ultimately toward device outlet 118 via the fluid flow path of the device. When a different flow rate is desired, the control knob can be conveniently rotated to bring another passageway 206 into communication with outlet 29b. To assist the caregiver in selecting a desired fluid flow rate, rate control graphics 207 are viewable through an opening 207a formed in the device housing (see FIGS. 1, 2, and 9).

Another important aspect of the invention resides in the provision of rate control locking means for preventing the unauthorized setting of rate controls by the rotation of driving member or control knob 190. This novel rate control locking means here comprises the previously identified key housing 158 as well as the generally vertically extending locking shaft 166. As previously mentioned, housing 158 is rotatably carried within first cavity 160 formed in extension member 60 (FIG. 16), which cavity includes a generally key-shaped opening 160a that is accessible from the bottom of extension 60 so that the physician's key 162 can be used to rotate the key housing in the manner previously described.

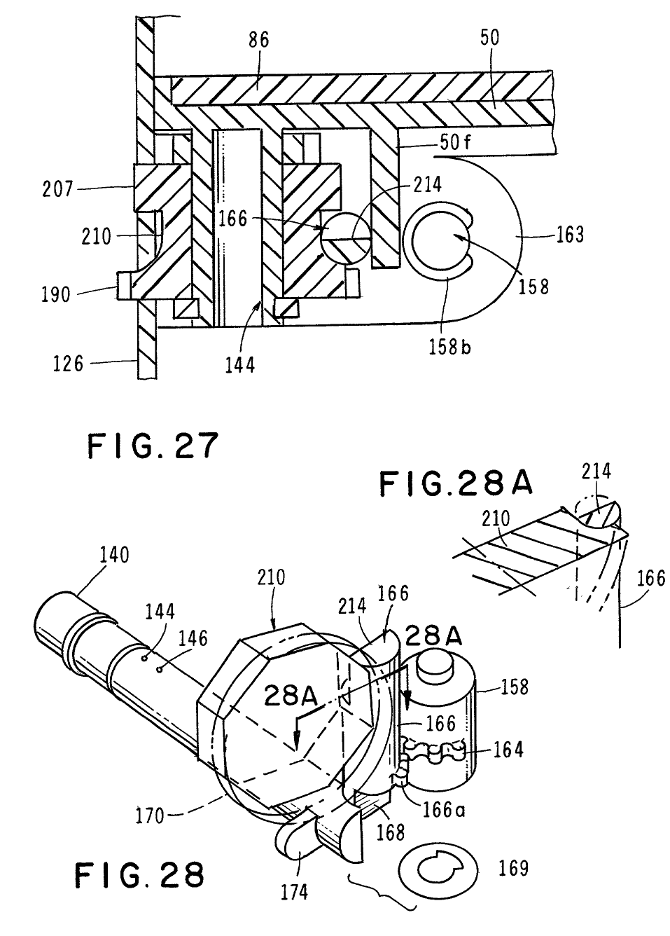

When locking shaft 166 is in the first normal position shown in FIGS. 27 and 28, the shaft positively prevents rotation of the control knob 180 by engaging an octagonal shaped member 210 that forms a part of control knob assembly 180 (FIGS. 28 and 28A). As previously discussed and as shown in FIGS. 24, 25, and 29, upon rotation of locking shaft 166 through an angle of 90 degrees in the direction of arrow 169 of FIG. 29 shaft 166 will move into a position that will permit operation of the diverter shaft 140. However, with the shaft in this second position, rotation of the control knob is still prevented thus preventing accidental resetting of the fluid flow rate. On the other hand, rotation of key housing 158 in the direction of the arrow 211 of FIG. 31, will cause the shaft 166 to move into the position shown in FIG. 31 where in a second flat 214 formed on the shaft moves into a position that will allow free rotation of the control knob in the direction of the arrow 215 (see also FIG. 30). As before, when the turning force exerted on the physician's key ceases, the arcuate coil spring 172 which circumscribes the key housing (FIGS. 24 and 26) will urge the key housing and the locking shaft 166 to tend to return to their starting positions thereby locking the control knob against further rotation.

Referring to FIG. 11, it is to be noted that indicia provided on the lower surface of cover 126 guides the caregiver in accomplishing the priming and rate control setting steps. For example, after the physician's key is inserted into opening 160a a rotation of the key in the direction of the counterclockwise arrow will move the locking shaft into the position shown in FIG. 29 permitting movement of the diverter shaft into the priming position. Similarly, rotation of the physician's key in the direction of the clockwise arrow of FIG. 11, will move the locking shaft into the position shown in FIG. 31 permitting rotation of the control knob 190 to set the desired rate of fluid flow to the patient. It is to be noted that the locking key cannot be removed until housing 158 and key 162 return to their initial starting position.

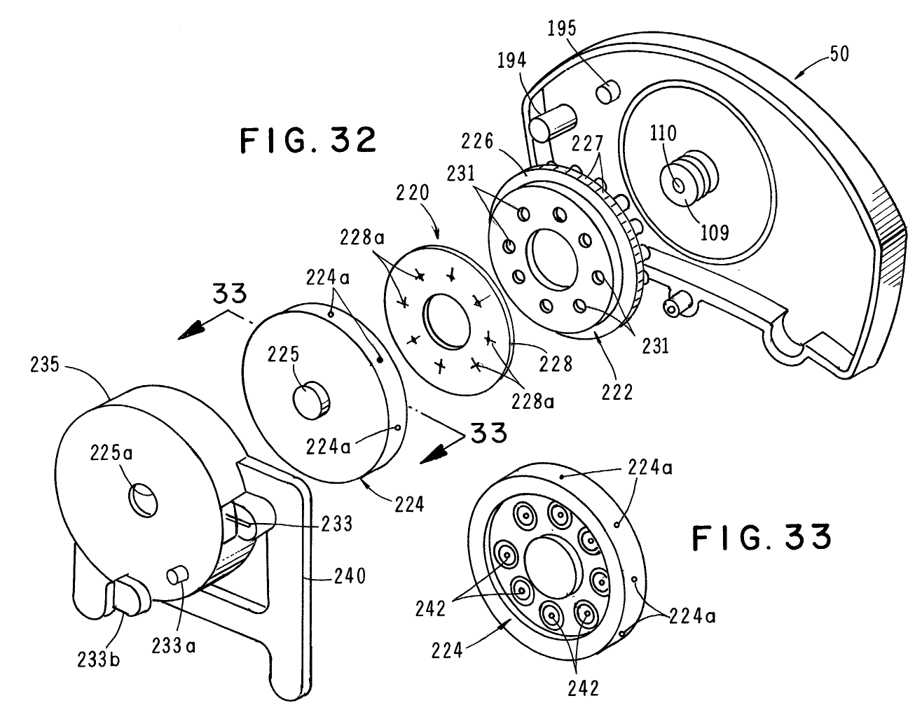

Turning to FIGS. 32 through 36 an alternate form of flow rate control means of the invention is there illustrated. This alternate means is similar in some respects to that previously described herein and like numerals are used to identify like components. As before, this alternate means comprises a rate control assembly that is rotatably mounted on hub 109 of superstructure 50 (FIG. 32). The alternate rate control assembly, here designated as 220, includes a rate control element base 222, a housing 224 and a back plate 226 having teeth 227 formed about its periphery. Assembly 220 is controllably rotated about hub 190 by a smaller diameter driving member, or toothed wheel (not shown) which is, in turn, driven by a finger engaging knob (not shown) both of which are of the character previously described. As before, the control knob rotates about a spindle 194, while the toothed wheel rotates about a spindle 195 to impart rotation to assembly 220 about hub 109. As best seen in FIG. 32, housing 224 is provided with a hub 225 that is rotatably supported within an aperture 225a formed in the flow rate control housing of the device, the character of which will presently be described.

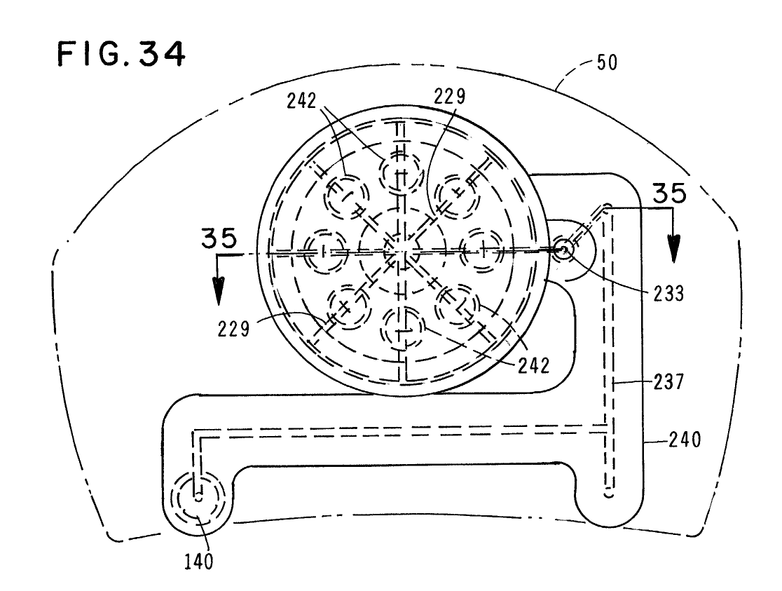

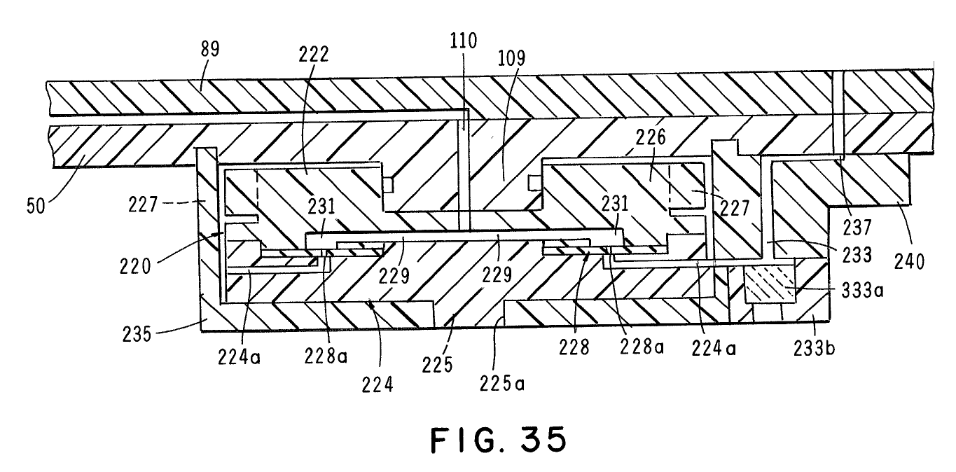

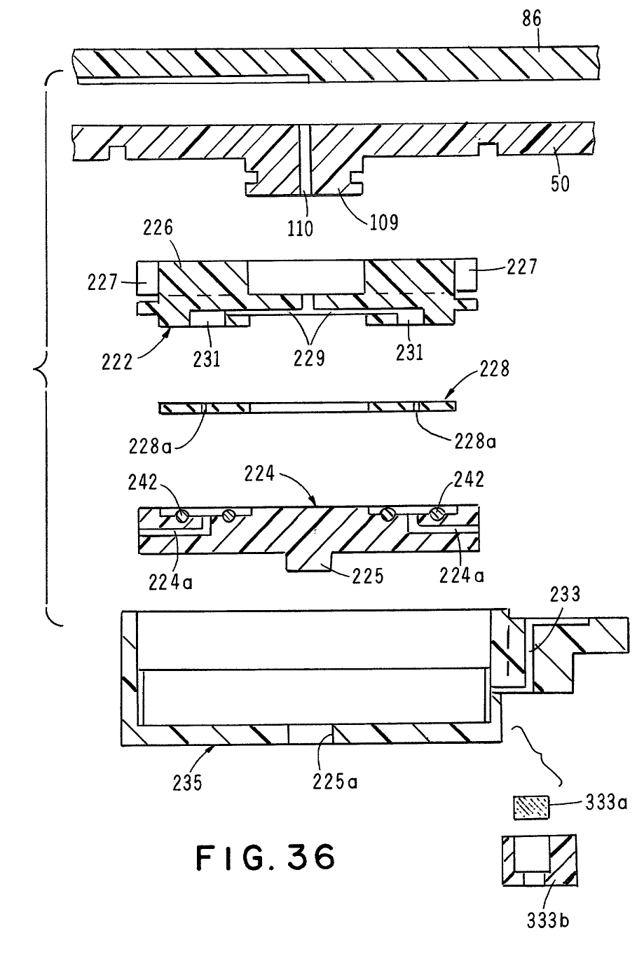

The primary difference between this latest form of flow rate control means of the invention and that earlier described resides in the replacement of the capillary type rate control elements with rate control elements comprising axially extending laser drilled microbores 228a formed in a rate control disc 228. As before base 222 is provided with a plurality of radially extending fluid passageways 229 (FIG. 34). However, in this instance, each of the passageways 229 communicates with a selected one of a plurality of spaced-apart apertures 231 formed in base 222 (FIG. 33). As best seen in FIGS. 35 and 36, rate control disc 228 is sealably disposed between base 222 and housing 224, the latter of which is provided with circumferentially spaced outlets 224a that communicate with a flow passageway 233 formed in a flow rate control housing 235 which is similar in construction and purpose to flow rate control housing 150 of the earlier described embodiment. Passageway 233 communicates with a passageway 237 formed in a member 240 that is received within superstructure 50. A plurality of circumferentially spaced O-ring elastomeric seals 242 are mounted on base 224 to prevent leakage of fluid internally of housing 224. As in the earlier described embodiment, fluid flowing through a selected rate control microbore will flow into passageway 233 and then toward boot 94 in the manner previously described. Vent means in the form of a porous hydrophobic vent 233a is provided (FIG. 35) and is held in position by a cover 233b. In operation, by rotating the control knob of the device, it is apparent that a selected passageway 229 and a microbore of a selected size can be positioned within housing 235 so as to communicate with passageways 233 and 237 and ultimately with device outlet 118. In this way the rate of fluid flow toward outlet 118 and then toward the patient can be precisely controlled. It is to be understood that the priming and locking functions previously described can be accomplished in the same manner with similar structure.

The fifth major subassembly of the invention, namely the infusion means for delivering the medicinal fluid to the patient is usable with both the previously described forms of the invention. This important means comprises a conventional delivery line 250 that is interconnected with the device outlet 118 in the manner shown in FIG. 1. In addition to the delivery line 250, the infusion means of the invention also includes a line clamp 252 which is of conventional construction and a gas vent and filter unit 254 which is also of a conventional construction well known to those skilled in the art.

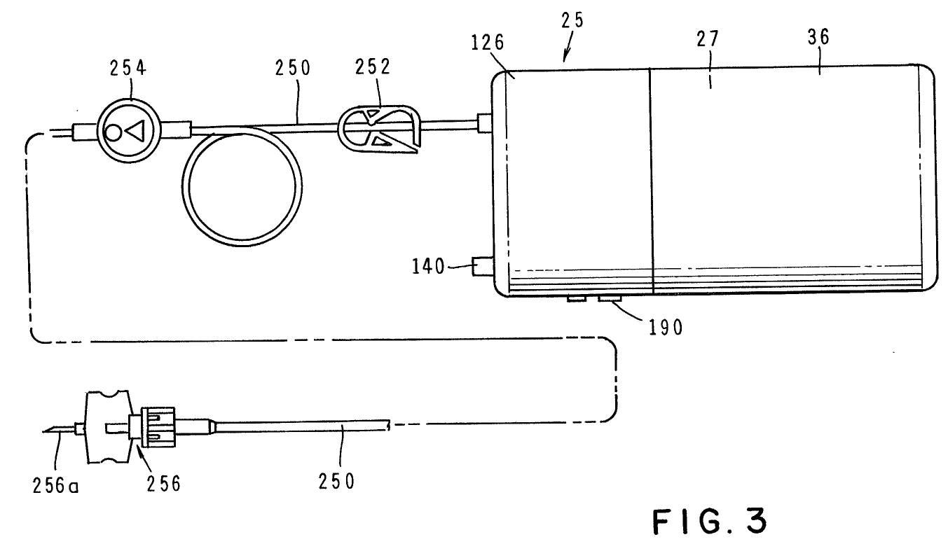

Once the adjustable flow rate control means of the invention has been set in the manner described in the preceding paragraphs and the fluid flow path has been primed, the infusion cannula 256a of the cannula assembly 256 of the invention (FIG. 3) can be invasively interconnected with the patient and the fluid delivery step can commence. During the delivery step, fluid will flow toward the patient at the rate of flow selected by the caregiver at the time of setting the fluid flow rate control means of the invention. In the manner previously described, the fluid status of the device can be continuously monitored by observing the various flow symbols of the indicator means that appear through viewing window 128 of the apparatus.

Having now described the invention in detail in accordance with the requirements of the patent statutes, those skilled in this art will have no difficulty in making changes and modifications in the individual parts or their relative assembly in order to meet specific requirements or conditions. Such changes and modifications may be made without departing from the scope and spirit of the invention, as set forth in the following claims.

We claim:

1. A fluid delivery device for dispensing fluid, said device comprising:
(a) a housing including a fluid passageway defining a flow path and having an inlet and an outlet;
(b) a fluid reservoir disposed within said housing in fluid communication with said inlet of said fluid passageway;
(c) stored energy means cooperatively associated with said fluid reservoir for urging fluid to flow therefrom toward said outlet of said fluid passageway; and
(d) flow rate control means carried by said housing intermediate said fluid reservoir and said outlet for controlling of the rate of fluid flow toward said outlet of said fluid passageway via said flow path, said flow rate control means comprising a control member having first and second spaced apart flow passageways that are selectively movable into communication with said flow path by movement of said control member from a first position wherein said first flow passageway is in communication with said flow path to a second position wherein said second flow passageway is in communication with said flow path, each of said first and second flow passageways having a flow rate control element operably associated therewith.

2. The device as defined in claim 1 in which each of said flow rate control elements comprises a flow rate control capillary mounted within said first and second flow passageways.

3. The device as defined in claim 1 in which each of said flow rate control elements comprise a laser drilled aperture in communication with said first and second flow passageways.

4. The device as defined in claim 1 in which said control member is rotatable about a longitudinal axis and in which said first and second flow passageways extend radially outward from said longitudinal axis.

5. The device as defined in claim 1 in which said housing includes a base and in which said stored energy means comprises a distendable member superimposed over said base, said member being distendable as a result of pressure imparted by the fluid to be dispensed to establish internal stresses, said stresses tending to move said member toward a less distended configuration.

6. The device as defined in claim 1 further including fill means carried by said base for filling said fluid reservoir with the fluid to be dispensed.

7. The device as defined in claim 1 further including a fluid actuated indicator means disposed intermediate said reservoir and said outlet of said fluid passageway for visually indicating fluid flow from said reservoir.

8. A fluid delivery device having an outlet comprising:
(a) a housing having a fluid flow passageway in communication with said outlet, said housing including a base;
(b) stored energy means for forming in conjunction with said base a fluid reservoir having a reservoir inlet and a reservoir outlet in communication with said fluid flow passageway, said stored energy means comprising a distendable member superimposed over said base, said member being distendable as a result of fluid introduced into said fluid reservoir to establish internal stresses within said member tending to return said member toward a less distended configuration to force fluids from said reservoir into said fluid flow passageway;
(c) fluid actuated indicator means in communication with said fluid flow passageway for visually indicating fluid flow from said reservoir; and
(d) adjustable flow rate control means in communication with said fluid flow passageway for controlling fluid flow from said outlet, said flow rate control means comprising a control member rotatably carried by said housing, said control member having first and second spaced apart radially extending flow passageways selectively movable into communication with said fluid flow passageway upon rotation of said control member, said first passageway having disposed therewithin a first flow rate control element and said second passageway having disposed therewithin a second flow rate control element.

9. The device as defined in claim 8 further comprising fill means carried by said base for filling said fluid reservoir.

10. The device as defined in claim 8 in which said first and second flow rate control elements comprise flow rate control capillaries.

11. The device as defined in claim 8 in which said adjustable flow rate control means further comprises a driving member mounted on said support for rotating said control member.

12. The device as defined in claim 11, in which said device further includes control knob operably connected to said driving member for a finger engaging, controllably rotating said driving member.

13. The device as defined in claim 11 further including rate control locking means for preventing rotation of said driving member.

14. The device as defined in claim 13 in which said rate control locking means comprises an elongated shaft carried by said housing for movement between a first, at-rest position, to a second locking position preventing rotation of said driving member.

15. The device as defined in claim 13 further including priming means for priming said fluid flow passageway, said priming means comprising:
(a) a bypass passageway formed within said housing for bypassing said flow rate control means, said bypass passageway having a first end in fluid communication with said fluid reservoir and a second end in communication with said fluid actuated indicator means; and
(b) diverter means for selectively directing fluid flow from said fluid reservoir along first and second paths, said first path permitting fluid to flow in a direction toward said flow rate control means and said second path permitting fluid flow into said bypass passageway, thereby by passing said flow rate control means.

16. A fluid delivery apparatus having an outlet comprising:
(a) a housing including a base and having a fluid flow passageway in communication with said outlet, said fluid flow passageway having communicating first, second and third segments;
(b) stored energy means for forming in conjunction with said base a fluid reservoir having a reservoir inlet and a reservoir outlet in communication with said first and second segment of said fluid flow passageway, said stored energy means comprising a distendable member superimposed over said base, said member being distendable as a result of fluid introduced into said fluid reservoir to establish internal stresses within said member tending to return said member toward a less distended configuration to force fluids from said reservoir through said reservoir outlet;
(c) fluid flow rate control means carried by said housing for controlling the rate of fluid flow from said reservoir toward said outlet of said housing, said fluid flow rate control means being in communication with said third segment of said fluid flow passageway and being in communication with said reservoir via said first segment of said fluid flow passageway;
(d) fluid actuated indicator means carried by said housing for indicating fluid flow from said reservoir, said indicator means being in fluid communication with said fluid rate control means and being in communication with said reservoir via said first, second and third segments of said fluid passageway; and
(e) priming means carried by said housing for selectively permitting fluid flow from said reservoir toward said outlet of said housing via said first, second and third segments of said fluid flow passageway and toward said outlet of said housing via said first segment of said fluid flow passageway, via said fluid flow rate control means and via said third segment of said fluid flow passageway.

17. The device as defined in claim 16 in which said fluid flow rate control means comprises a rotatably mounted control member having first and second circumferentially spaced-apart flow passageways that are selectively movable into communication with said first segment of said fluid flow passageway by rotation of said control member from a first position wherein said first flow passageway is in communication with said first segment to a second position wherein said second flow passageway is in communication with said first segment, each of said first and second flow passageways having a flow rate control element disposed therewith.

18. The device as defined in claim 16 further including fill means carried by said base for filling said fluid reservoir with the fluid to be dispensed.

19. The device as defined in claim 16 further including infusion means connected to said housing in communication with said outlet for delivering the fluid to a patient.

20. The device as defined in claim 16 in which said flow rate control means comprises a rotatably mounted control member and in which said device further includes flow rate control locking means for preventing rotation of said control member.

21. The device as defined in claim 16 in which said priming means comprises a diverter shaft carried by said housing for movement between a first, at rest position wherein fluid flows from said reservoir toward said fluid flow rate control means and a second fluid diverting position wherein fluid flowing from said reservoir is diverted into said second segment of said fluid flow passageway thereby bypassing said fluid flow rate control means.

22. The device as defined in claim 21 in which said diverter shaft includes a fluid passageway which, when said diverter shaft is in said second fluid diverting position, directs fluid flow from said reservoir through said first segment into said second, bypass segment.

23. The device as defined in claim 21 further including diverter shaft locking means for preventing movement of said diverter shaft into said second diverting position.
